(19) United States
(12) Patent Application Publication
(10) Pub. No.: US 2024/0371495 A1
(43) Pub. Date: Nov. 7, 2024

(54) MEDICAL SYSTEM AND METHOD FOR PROVIDING A TREATMENT PROPOSAL

(71) Applicant: Aesculap AG, Tuttlingen (DE)

(72) Inventors: Ronja Alissa Schierjott-Hermle, Tuttlingen (DE); Georg Hettich, Freiburg (DE)

(21) Appl. No.: 18/681,055

(22) PCT Filed: Aug. 4, 2022
(86) PCT No.: PCT/EP2022/072018

§ 371 Date: Feb. 4, 2024

(30) Foreign Application Priority Data
Aug. 5, 2021 (DE) 10 2021 120 380.5

(51) Int. Cl. G16H 20/40; A61B 34/10; G16H 10/60; G16H 15/00; G16H 50/70

(52) U.S. Cl. CPC G16H 20/40; A61B 34/10; G16H 10/60; G16H 15/00; G16H 50/70; A61B 2034/104; A61B 2034/108

(57) ABSTRACT

A medical system and method can be used to determine a treatment suggestion when treating a bone with an implant. In a memory device there are stored treatment data records for previous treatment interventions, containing parameter information, treatment information and success information. A data processing device determines, for an upcoming treatment intervention, at least one selected treatment kind with respect to a successful treatment intervention based on parameter information and depending on the linking of the success information with the parameter information of previous treatment interventions. A treatment proposal regarding the at least one selected treatment kind is output to the user.

### CROSS-REFERENCE TO RELATED APPLICATIONS

This application is the United States national stage entry of International Application No. PCT/EP2022/072018, filed on Aug. 4, 2022, and claims priority to German Application No. 10 2021 120 380.5, filed on Aug. 5, 2021. The contents of International Application No. PCT/EP2022/072018 and German Application No. 10 2021 120 380.5 are incorporated by reference herein in their entireties.

### FIELD

The present disclosure relates to a medical technology system for use in a treatment of a bone with an implant.

Furthermore, the present disclosure relates to a method for providing a treatment suggestion for an upcoming treatment of a bone with an implant.

The medical system and the method are used in particular in an implantation of an artificial acetabulum into a human pelvic bone.

Examples of applications include revision surgeries, in which an existing implant, in particular an artificial acetabulum in a pelvic bone, is removed and replaced with a new implant.

### BACKGROUND

In the case of treatment interventions on bones, in particular pelvic bones, it is known to describe the condition of the bone before the intervention. ‘Treatment intervention’ can be regarded in particular as the aforementioned treatment of a patient with an implant. In particular, the treatment intervention may include preparatory steps such as an analysis of the actual state and/or planning, the surgical intervention in the actual sense, and preferably a follow-up treatment or analysis of the postoperative state.

During treatment, for example, bone defects are identified, on the basis of which the surgeon has to decide which implant and/or which surgical technique to use. It is known to classify the bone defects and/or to describe the bone defects segment by segment.

DE 10 2018 116 588 A1 describes a medical technology toolset and a corresponding method. An actual-state data set of a bone that is considered defective is created. A healthy-state data set and a planning data set are calculated on the basis of the healthy-state data set. The surgeon's instructions for treating the bone are incorporated into the creation of the planning data set, which includes, for example, information about characteristic anatomical features of the bone. The planning data set can be shown to the surgeon on a display device. The toolset comprises a medical navigation system and a marking device that can be defined on the bone to define a reference. The planning data set can be displayed on the display device in spatial relation to the bones, whereby characteristic landmarks of the bone are assigned to characteristic landmarks in the planning data set.

### SUMMARY

The object of the present disclosure is to provide a medical system and a method for providing a treatment suggestion that supports a user, in particular a surgeon, in the treatment of the bone.

According to the present disclosure, the object is solved by a medical technology system (or medical device/apparatus) and in particular by a medical system (or medical device/apparatus) for use in a treatment of a bone with an implant, in particular in an implantation of an artificial acetabulum into a human pelvic bone, especially for revision interventions, wherein the system comprises or has a data processing device, a memory device and a notice device, wherein in the memory device, based on a plurality of preceding treatment interventions, the following is stored linked to each other and assigned or assignable to a respective treatment intervention in an (electronically readable) treatment data set:
parameter information comprising a plurality of objectifiable parameters that are indicative of a pathological condition of the pelvic bone,
treatment information that is indicative of a conducted treatment type for the patient for the treatment intervention, from a group of preferably predefined treatment types, and
success information that is indicative of the success of the treatment intervention,
wherein the data processing device is configured and provided such that it determines (relevant) parameters for (individual) treatment types (of a plurality of preceding treatment interventions) from the treatment data set via a static model, which are decisive for the success of the respective treatment type. Furthermore, the data processing device, for an upcoming treatment intervention, compares parameters for the upcoming treatment intervention with the determined (relevant) parameters of the plurality of preceding treatment interventions in order to determine at least one selected treatment type from the group of treatment types with respect to a successful treatment intervention, and provides a treatment suggestion about the at least one selected treatment type to a user at the notice device.

Preferably, a deviation between the parameters of the upcoming treatment intervention and the parameter information of the (historical) treatment data set is calculated, wherein in particular a deviation between the parameters of the upcoming treatment intervention and the determined relevant parameters of the treatment data set is calculated. This deviation should preferably lie within a predetermined tolerance range. Thus, a quantity of preceding treatment interventions can be determined, which are similar in the relevant parameters to the parameters of the upcoming treatment intervention. A treatment type of a successful preceding treatment intervention with similar relevant parameters can thus be determined as a promising/likely successful treatment type for the upcoming treatment intervention.

The medical system or apparatus for use in a treatment of the bone with the implant, especially for revision interventions, comprises the data processing device, the memory device and the notice device. In the memory device, based on the plurality of preceding treatment interventions, the following is stored linked to each other and assigned or assignable to a respective treatment intervention in a treatment data set:
the parameter information comprising a plurality of objectifiable parameters indicative of a pathological condition of the bone;
the treatment information that is indicative of a conducted treatment type for the patient (52) for the treatment intervention, from a group of preferably predefined treatment types; and
the success information that is indicative of the success of the treatment intervention,
wherein the data processing device contains the following method steps in electronically readable form:
determination of relevant parameters from the treatment data set for the respective treatment types by a static model, in particular by a principal component analysis, wherein the relevant parameters are decisive for the success of the respective treatment type;
comparing, for an upcoming treatment intervention, the parameters of the upcoming treatment intervention with the determined relevant parameters of the plurality of preceding treatment interventions, wherein a deviation between the parameters of the upcoming treatment intervention and the determined relevant parameters lies within a predetermined tolerance range;
selecting at least one (likely successful) treatment type from the group of treatment types with regard to a successful treatment intervention; and
outputting the at least one selected treatment type to a user through the notice device.

The data processing device may further cause an executing unit, in particular a CPU, to (electronically) execute the above method steps.

In other words, according to the disclosure, the data processing device is configured and adapted (programmed) such that it determines relevant parameters for treatment types from the treatment data set by a static model, in particular principal component analysis, wherein the relevant parameters are decisive for the success of the respective treatment type, and it compares parameters of the upcoming treatment intervention with the determined relevant parameters of the plurality of preceding treatment interventions for an upcoming treatment intervention, wherein (in particular) a deviation between the parameters of the upcoming treatment intervention and the determined relevant parameters lie within a predetermined tolerance range, in order to determine at least one selected treatment type from the group of treatment types with respect to a successful treatment intervention, and to provide a treatment suggestion about the at least one selected treatment type to a user at the notice device.

In the medical technology system according to the disclosure, information about preceding treatment interventions may be stored. In the present case, ‘treatment intervention’ may in particular be a single, customizable treatment of a patient who has been implanted with an implant, in particular an artificial acetabulum in the pelvic bone. A treatment data set is stored in the memory device for each treatment intervention. The treatment data set comprises the parameter information, which in particular describes a preoperative state of the bone, the treatment information about the treatment type performed on the patient, and the success information, which in particular was obtained postoperatively.

The disclosure includes the consideration that experience gained during preceding treatment interventions can be utilized for the upcoming treatment intervention on another patient as well as for future treatment interventions and can be used by the system, in particular automatically, to provide the treatment suggestion. The parameter information comprises a plurality of objectifiable parameters, which are, for example, an output of a quantitative bone defect analysis. The data processing device is configured and programmed to determine at least one selected treatment type from the group of possible treatment types based on the empirical values of preceding treatment interventions stored in the treatment data sets. The at least one selected treatment type is suggested to the user via a corresponding treatment suggestion provided at the notice device. The user can accept the treatment suggestion submitted to them and can carry out the treatment intervention based on this. Alternatively, it may be provided that the user can choose a different treatment type from the group of treatment types in deviation from the treatment suggestion. In this way, the experience gained by surgeons on the basis of the success information for a selected treatment type based on the predefined parameter information can be used advantageously for future treatment interventions and can support the user in these treatment interventions.

In the present case, the user may in particular be a natural person, in particular a surgeon. Alternatively, the user may be or comprise, for example, a data processing device to which the treatment suggestion is output and which, for example, controls and/or performs the treatment intervention at least partially in an automated and/or manual manner controlled by an operator, for example in the sense of a human-machine interface.

The treatment data sets may be stored in the memory device in different ways. In particular, it is not necessary for an independent file to be assigned to a respective treatment data set, although this may be possible. The parameter information, the treatment information and the success information of the different treatment interventions may, for example, be stored together in a database in such a way that the respective information, which is linked to each other and to the treatment intervention, can be identified at any time. For example, the treatment data sets are stored in matrix form, for example via relations in a relational database.

It may be provided that the treatment data sets can be stored centrally in the memory device or in a decentralized manner, wherein the memory device may be configured in a distributed manner.

It is favorable if the system comprises an analysis device for providing an initial data set that is indicative of an actual state of the bone, wherein the data processing device is configured and programmed to determine at least part of the parameters for providing the parameter information on the basis of the initial data set. In this way, parameters for the parameter information can be obtained preoperatively. For example, the initial data set is transferred from the analysis device to the data processing device via a communication interface. In particular, the data processing device may analyze the initial data set and, for example, may perform a segmentation of the bone for the purpose of a quantitative bone defect analysis.

The analysis device is or comprises, for example, an X-ray and/or CT device, wherein the initial data set is or comprises an X-ray image and/or a CT data set of the actual state.

Favorably, the data processing device is configured and programmed to computationally remove an implant existing in the bone from the initial data set for the actual state of the patient. During revision surgery, an existing implant is removed and replaced by a new implant. For this purpose, it is advantageous if the initial data set of the data processing device is available for analysis free of an implant (i.e. a contribution in the initial data set that goes back to the implant).

The data processing device is preferably configured and programmed to computationally determine a healthy-state data set of the patient based on the initial data set.

Preferably, it is provided that the data processing device determines at least a part of the parameters by comparing the healthy-state data set with the initial data set or, if available, the initial data set after computational removal of the implant as explained above. The healthy-state data set is created, for example, with the aid of a statistical form model in which the statistically most probable healthy situation of the bone (native situation) is reconstructed. A quantitative bone defect analysis can then be carried out, for example, by comparing the ‘healthy-state data set’ and ‘initial data set’ situations, possibly without an implant.

The initial data set and/or the healthy-state data set includes, for example, a 3D representation of the bone.

The parameters of the parameter information may, for example, include at least one of the following:
relative and/or absolute bone volume loss in at least one portion of the bone;
relative and/or absolute new bone formation in at least one portion of the bone;
ovality of the acetabulum in a pelvic bone;
center-notch angle of the acetabulum in a pelvic bone;
implant migration in the bone as a function of time;
wall defects of the bone;
available support surface for an implant.

The groups of treatment types differ from each other, for example, by one or more of the following:
type of implant;
implantation technique;
use of a standardized implant;
use of a patient-specific implant;
use of separate anchoring elements to fix the implant;
use of bone replacement material.

If, as mentioned at the beginning, groups of treatment types are provided (also described as ‘treatment manner’), these may differ from each other in particular on the basis of technically available and/or used solutions. Using the example of hip revision surgery, the following treatment types may be provided:
a) press-fit cup as a hemispherical, cement-free cup that is fixed by force-fit;
b) press-fit cup with augmentation as a cement-free hemispherical cup, which has to be additionally provided with an augmentation and screw fixation due to a bone contact area of less than 50%;
c) support shell as defect bridging element to be screwed together in case of insufficient contact surface in the remaining bone;
d) patient-specific customization.

It goes without saying that the above examples only reflect the different treatment types as examples.

Advantageously, the data processing device is configured and programmed to determine the at least one selected treatment type linked with a probability for a successful treatment intervention and that the treatment suggestion to the user comprises an indication of the probability. In this way, the user receives further valuable information on the basis of which they can decide on the at least one treatment type.

For example, the probability may be specified as a threshold probability, wherein the treatment success occurs above a lower probability threshold or up to an upper probability threshold. Alternatively or additionally, it is conceivable to specify the probability as a probability interval within which the treatment success occurs.

The data processing device is preferably configured and programmed to determine two or more selected treatment types from the group of treatment types and to provide the treatment suggestion about the two or more selected treatment types to the notice device. For example, if the data processing device determines that, based on the parameter information for the treatment intervention to be performed, more than one treatment type is likely to be successful, the treatment suggestion may comprise the indication of two or more treatment types.

In the latter case, the treatment suggestion preferably includes an indication of the respective probability of a successful treatment intervention for at least two of the selected treatment types. This supports the user in selecting the treatment type for the treatment intervention to be carried out.

It is favorable if the data processing device is configured and programmed to create a quantitative relationship between characteristic features of a respective treatment type and at least part of the parameters of the parameter information, preferably selected parameters, for in particular preceding treatment interventions. In particular, the quantitative relationship may be or form a static model in which characteristic features of bone defects are identified within each treatment type and are related to the parameters. For this purpose, the data processing device may, for example, weight the parameters differently.

For example, an executable and feasible algorithm may be stored in the data processing device in order to create the quantitative relationship. The algorithm may be used to identify characteristic features, for example via a PC analysis (PCA, Principal Component Analysis).

For example, the use of a machine learning algorithm and/or a neural network of the data processing device to create the quantitative relationship is favorable.

It is advantageous if the data processing device is configured and programmed to include the success information in this quantitative relationship for the respective treatment intervention and uses this quantitative relationship as the basis for determining the at least one selected treatment type.

For example, if a previous treatment intervention was successful, a set of parameters can be identified that match parameters of the upcoming treatment within a predefined or predeterminable threshold. These parameters may, for example, be identified as predominantly decisive in the determination of the treatment type, so that characteristic features of the treatment type can be derived from this. Based on previous experience, it is then possible to suggest the, at least one, best possible treatment type.

The data processing device is preferably configured to be portable additional device and programmed to create the quantitative relationship without user intervention.

Treatment data sets of new treatment interventions are preferably stored in the memory device and are taken into account for future treatment suggestions.

The system preferably comprises at least one input device for receiving data input from a user after completion of the treatment intervention. Preferably, the data input is not provided immediately after completion, but after a predetermined or predeterminable acclimatization time of the patient.

The data processing device is preferably configured and programmed to create the success information based on the data input and to store it together with the parameter information and the treatment information in the treatment data set.

In a preferred embodiment of the disclosure, the input device may be at least one portable additional device arranged spatially remote from the data processing device, or the at least one additional device comprises the input device, wherein a user application program is stored executably on the additional device, and wherein the data input of the user can be transmitted from the additional device to the data processing device via a communication connection. The user application program may, for example, be a so-called app that is stored to be executable on the additional device. The additional device is, for example, a smartphone or a tablet computer. Inputs on the additional device are transmitted to the data processing device, which then creates and saves the success information.

In particular, the above embodiment offers the advantage that the patients themselves can contribute to the maintenance and refinement of the system using the user application program without the need for an operator.

The user data set may be subsequently changed, in particular with regard to the success information. For example, data inputs for the success information can be replaced and/or supplemented if the assessment of the success of the applied treatment type changes over time.

In a preferred embodiment of the disclosure, the system may comprise a stockage device in which a plurality of medical device toolsets are stored, each comprising at least one implant, wherein provision information is transmitted from the data processing device to the stockage device in response to the treatment suggestion for providing the at least one toolset. The transmission of the provision information may be transmitted spontaneously by the data processing device without the user's intervention. Alternatively, it is conceivable that the user triggers the transmission of the provision information after selecting the treatment type. In the currently preferred embodiment, it is ensured that the toolset comprising the appropriate implant is provided for the treatment type. Information in this regard may be stored in the treatment data set, for example. In addition to the implant, the toolset may comprise, for example, an insertion tool and/or other surgical instruments.

It is conceivable that the stockage device automatically issues a request for the subsequent delivery of a toolset if this has been requested via the provision information.

It may be provided that the data processing device is configured centrally or that the data processing device is provided or formed by a computer network. For example, a decentralized data processing device may be used, which is implemented via a cloud.

As mentioned at the beginning, the present disclosure also relates to a method.

A method according to the disclosure for providing a treatment suggestion for an upcoming treatment of a bone with an implant, in particular using a medical system of the aforementioned type, comprises:
providing a treatment data set assigned or assignable to a respective treatment intervention for preceding treatment interventions in a memory device, in which the following is stored in a linked manner:
parameter information, comprising a plurality of objectifiable parameters that are indicative of a pathological condition of the bone;
treatment information that is indicative of a treatment type of the patient at the treatment intervention, from a group of preferably predefined treatment types; and
success information that is indicative of the success of the treatment intervention;
determining at least one selected treatment type from the group of treatment types for the upcoming treatment intervention, with a data processing device, based on the parameter information provided therefor and depending on the linking of the success information with the parameter information of a plurality of preceding treatment interventions, with respect to a successful treatment intervention; and
providing a treatment suggestion via the selected treatment type.

The disclosure further relates to a computer-implemented (pre) selection method or suggestion method. The method comprises the following steps:
extracting or reading, by a data processing device, of a treatment data set of preceding or historical treatment interventions from a memory device, wherein the treatment data set comprises parameters or parameter information (in particular width of a femur), treatment information (in particular intervention type and/or implant type) and/or success information (in particular a binary success parameter: yes/no) of the preceding treatment interventions;
determining, by the data processing device, of (relevant) parameters of respective treatment types which are decisive for the success (in particular success: yes) of the respective treatment type (in particular determining a correlation between successful treatment type and a selection quantity of (relevant) parameters which significantly influence the successful treatment type), from the treatment data set by a static model, in particular by a principal component analysis;
reading of an actual-state data set (initial data set) by the data processing device, in particular a CT image of a patient with parameters;
calculating, on the basis of the actual-state data set (initial data set), a target-state data set (healthy-state data set) by the data processing device, in particular with the help of a molded bone model;
determining differences or deviations of parameters/parameter information by comparing the target-state data set with the actual-state data set by the data processing device;
filtering of the differences or deviations with regard to the determined relevant parameters by the data processing device;
comparing the filtered differences of the parameters with those corresponding to the historically successful treatment interventions, and determining a quantity of historical treatment data for which the differences are similar to each other (in particular within a tolerance range, e.g. for which a sum of the relative differences of the relevant parameters is as small as possible);
outputting by a notice device, in particular a display, the determined quantity of historical successful treatment data, in particular the treatment information.

In particular, the treatment information may include an implant to be used in the treatment and/or an instrument to be used in the implantation of the implant.

In the step of determining the (relevant) parameters of the historical treatment data set, a quantitative relationship between the parameter information of the individual historical treatment interventions and the success information of the historical treatment interventions is preferably determined. It can be determined which parameters are decisive for the success of a treatment intervention. The relevant parameters may also be characteristic features for the success of a treatment intervention. The quantitative relationship may be determined by a static model, in particular a principal component analysis (PCA), or a machine learning algorithm and/or a neural network. This step can thus in particular determine a set of parameters that are relevant for the success of the treatment intervention.

In the step of reading in the actual-state data set, an initial data set of the bone or of the bone with the (defective) implant is preferably read in. The initial data set may be a CT data set or an X-ray image with a 3D representation of the bone.

In the step of calculating the target-state data set from the actual-state data set, the target-state data set, which is a healthy-state data set, is preferably calculated by a statistical form model for the bones. Preferably, a statistically probable healthy situation of the bone is calculated.

By comparing the target-state data set (healthy-state data set) with the actual-state data set (initial data set), the parameters or parameter information can preferably be determined. In particular, this may be a difference between the target-state data set and the actual-state data set. The parameters may be at least partially objectifiable in order to be able to detect bone defects quantitatively.

In the next step, the parameters determined by comparing the target-state data set with the actual-state data set may be filtered so that only those parameters are considered that were classified as relevant based on the historical treatment data set, in particular by the statistical model. In other words, only the values of the relevant parameters may be considered.

In the next step, the differences between the target-state data set and the actual-state data set, i.e. the determined parameters of the above-mentioned treatment intervention, are compared with the parameter information of the historical treatment data set. In particular, only the relevant parameters are compared, i.e. only the filtered parameters.

In particular, a quantity of historical treatment data is determined in which the parameter information is similar to the corresponding parameter information of the upcoming treatment intervention. In this context, similar means that a difference between the individual parameters lies within a predetermined tolerance range. In particular, the quantity of historical treatment data may be a quantity of treatment types from the historical treatment data in which the parameter information (of the relevant parameters) is similar to the parameters of the upcoming treatment intervention. In particular, the quantity of historical treatment data may be limited to successful historical treatment interventions.

The specific quantity of historical treatment data may therefore be a quantity of treatment types that have a particularly good chance of success for the determined parameters. Thus, the determined treatment types may be particularly promising.

A sum of deviations (for the relevant parameters) may also be determined and a treatment type may be classified as particularly promising if the sum of deviations is particularly small.

The specified quantity of historical treatment data may be output to a user in a final step. The user can then receive a qualified suggestion (for a treatment type from the treatment data).

The determined parameters and a success/failure of a conducted treatment intervention can be added to the treatment data to expand the data set.

Preferably, the treatment information may comprise a group of treatment types for a treatment intervention, the parameter information may comprise parameters for the treatment interventions and the success information may comprise a success of the preceding treatment interventions. The treatment data set may link the respective treatment types with the parameters and the success of the treatment intervention.

The object of the present disclosure is further solved by a computer-readable storage medium comprising commands which, when executed by a computer, cause the computer to execute the method steps of the method according to one of the preceding aspects.

The advantages already mentioned in connection with the explanation of the system according to the disclosure can also be achieved when using the method. Reference is made to the above-mentioned explanations.

### BRIEF DESCRIPTION OF THE DRAWINGS

Advantageous configuration examples of the method according to the disclosure result from advantageous embodiments of the system according to the disclosure. Reference can also be made in this respect to the preceding explanations.

The following description of a preferred embodiment of the disclosure, in conjunction with the drawing, serves to explain the disclosure in more detail.

FIG. 1 shows a schematic representation of a medical system according to the disclosure in a preferred embodiment;

FIG. 2 shows a schematic representation of a memory device of the system shown in FIG. 1;

FIG. 3 shows a schematic representation of a stockage device of the system shown in FIG. 1;

FIG. 4 shows a representation of a bone considered to be pathological with an implant, in particular a pelvic bone with an artificial acetabulum, which is to undergo revision surgery;

FIG. 5 shows a schematic representation illustrating the operation of the system shown in FIG. 1;

FIG. 6 shows a schematic representation of a 3D representation of the pelvic bone from FIG. 4 with the implant computationally removed;

FIG. 7 shows a schematic representation of a computationally determined healthy state of the pelvic bone in a 3D representation;

FIG. 8 shows a schematic representation showing the operation of the system during an upcoming treatment intervention; and

FIG. 9 shows a flow diagram of a method according to the disclosure.

### DETAILED DESCRIPTION

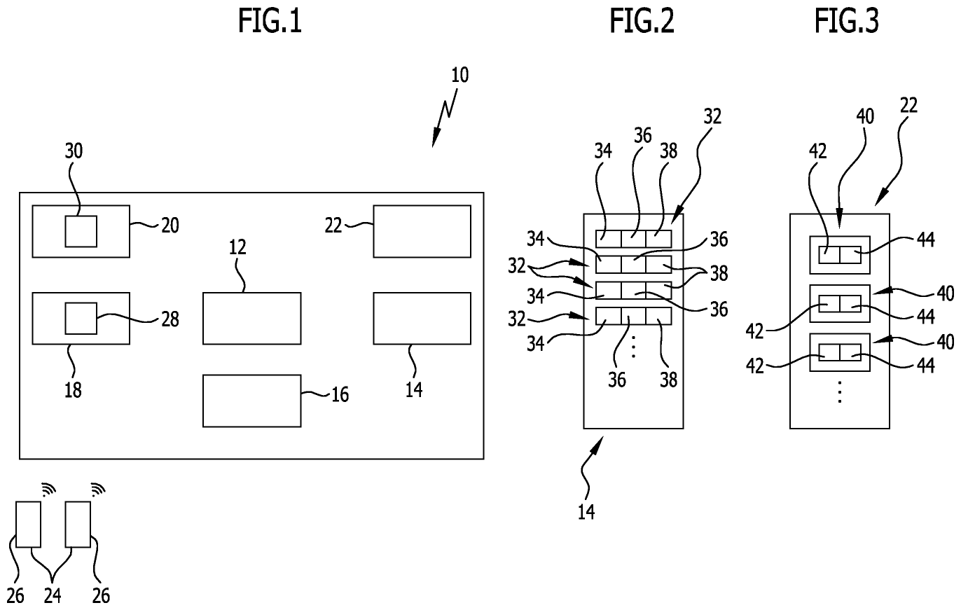

FIG. 1 shows a schematic representation of a generally advantageous embodiment of the medical system according to the disclosure with the reference sign 10. The system 10 is suitable for carrying out the method according to the disclosure and advantageous configuration examples of the method. A method procedure is described below in connection with the explanation of the operation of the system 10.

The system 10 comprises a data processing device 12, a memory device 14, an input device 16, a notice device 18, an analysis device 20 and a stockage device 22. The aforementioned components of the system 10 may be positioned centrally, at the same location, or spatially distributed.

For example, the data processing device 12 may be centrally configured and positioned, for example as a computer. Alternatively, the data processing device 12 may be configured by spatially distributed computers or servers, for example using a cloud service.

The same applies to the memory device 14, which may be configured centrally or spatially distributed. The memory device 14 may be integrated into the data processing device 12 or may be included in it.

It may also be provided, in particular, that the analysis device 20 and/or the stockage device 22 are configured and positioned at a distance from other components of the system 10, in particular the data processing device 12.

Further input devices 24 may be in operative connection with the system 10, in particular the data processing device 12, and in particular may be coupled via a communication connection in terms of information technology. The input devices 24 are configured as portable additional devices 26. FIG. 1 shows two such additional devices 26. The input devices 24 may be part of the system 10.

The components of the system 10 can be wired and/or wirelessly coupled or connectable to each other in order to exchange information.

The input devices 16 and/or 24 may be designed in different ways and may comprise, for example, a keyboard, a computer mouse, a touch screen and/or other input means. Voice input is also conceivable, for example.

The notice device 18 is or comprises, in particular, an optical display unit 28 for outputting visual notices to a user.

In the present configuration example, the analysis device 20 is or comprises, for example, a CT device 30.

The memory device 14 stores treatment data sets 32 of treatments of bones of patients for which the system 10 was used for data acquisition and data management.

Each treatment data set 32 is assigned to a respective treatment intervention using the system 10. A respective treatment data set 32 comprises, for a preceding treatment intervention, parameter information 34, a treatment information 36 and a success information 38 (FIG. 2).

Here, the representation of FIG. 2 is schematic. The information 34, 36, 38 may be stored in the respective treatment data set 32 in different ways. For example, the treatment data sets are stored in matrix form, for example in a relational database, wherein FIG. 5, explained below, schematically illustrates an exemplary matrix representation of the contents of the treatment data sets 32.

The stockage device 22 shown schematically in FIG. 3 is used for stocking and managing medical toolsets 40. A respective toolset 40 comprises, in particular, an implant 42 to be used in the treatment of the bone, in particular an artificial acetabulum. In addition, each toolset 40 may comprise, for example, at least one surgical instrument 44 to be used during implantation of the implant 42.

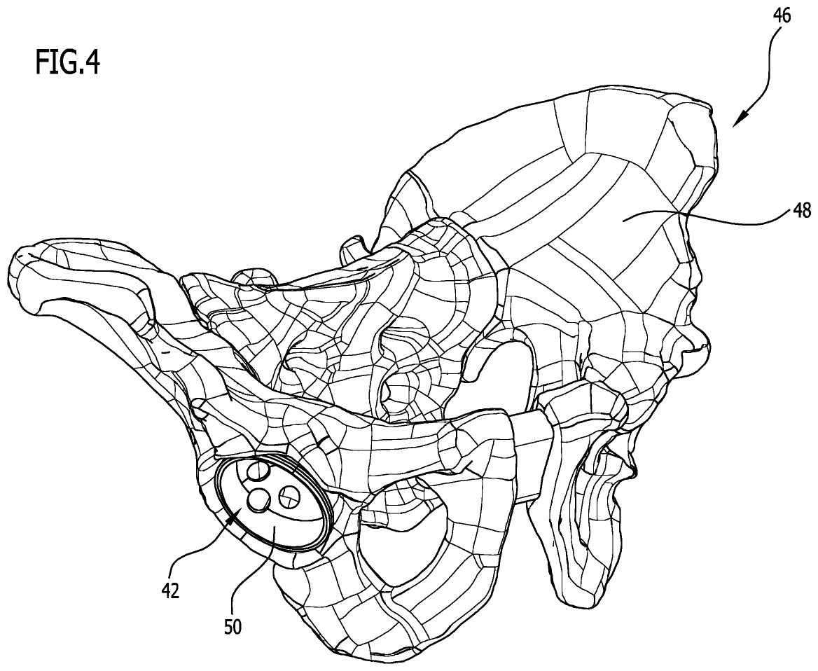

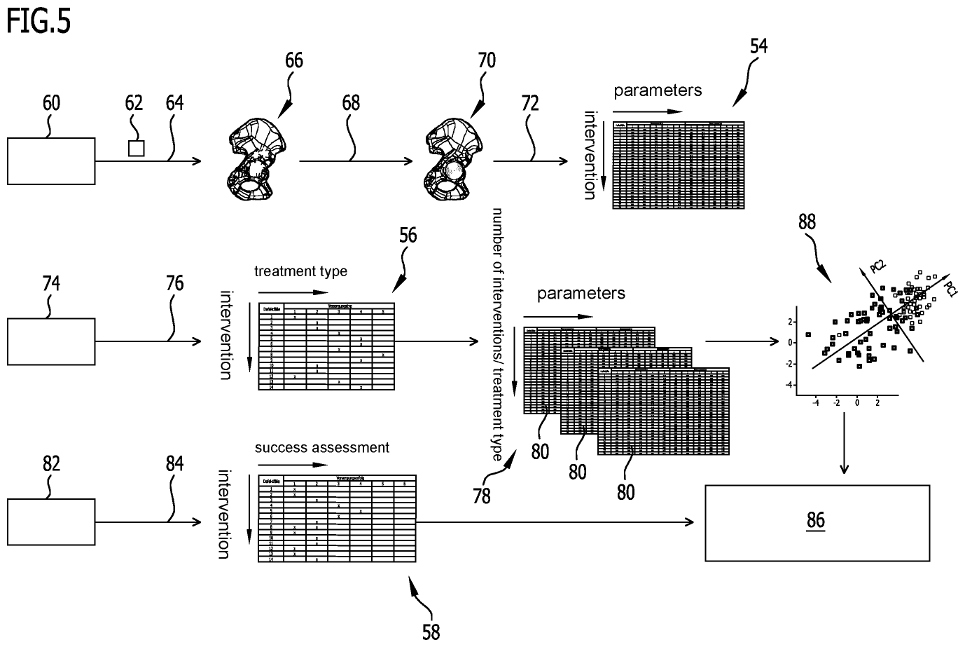

FIG. 4 shows a perspective view of a bone 46 to be treated, designed as a human pelvic bone 48. An implant 42 is inserted in the pelvic bone 48, in this case an artificial acetabulum 50. As mentioned, the system 10 is used, for example, for treatments of the pelvic bone 48, which is considered to be pathological and in particular has bone defects. In this case, the implant 42 is removed in revision surgery and replaced by a new implant.

It is to be understood that the present disclosure is not limited to use in revision surgeries and is not limited to use in treatments of a pelvic bone 48.

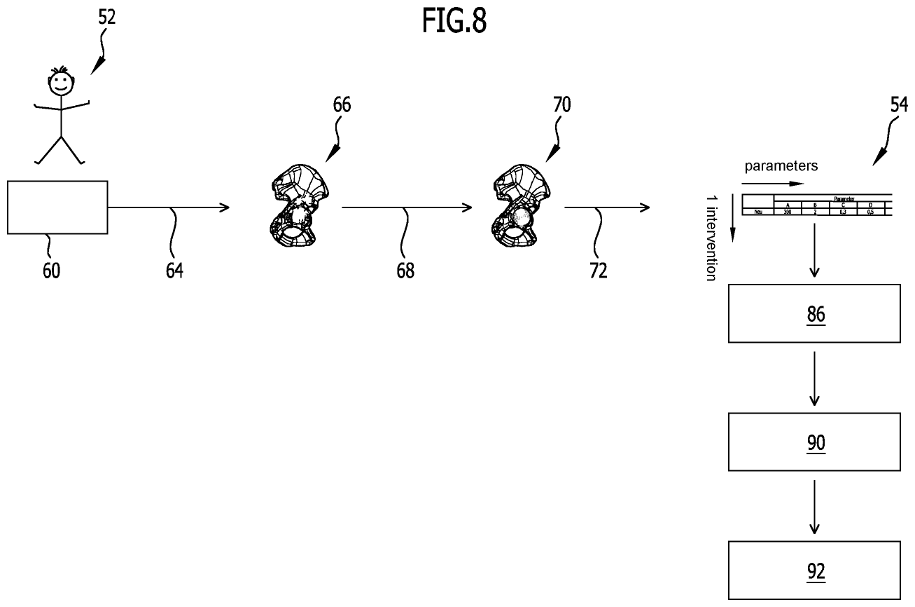

For an upcoming treatment intervention, which is explained below with reference in particular to FIG. 8, information stored in the treatment data sets 32 is evaluated by the system 10 to suggest a treatment type for the treatment of the patient 52.

For the functional principle of the system 10, reference is first made to FIGS. 5 to 7.

The system 10 uses the treatment data sets 32 of a plurality of preceding treatment interventions to submit a treatment suggestion for a treatment type for the treatment of the patient 52. It is advantageous if a large number of treatment interventions is stored in the system 10 and can be utilized by it. For example, at least approximately 50 treatment interventions are advantageous, more preferably at least approximately 200 treatment interventions, with a higher number being preferred. The parameter information 34, the treatment information 36 and the success information 38 are taken into account in each case.

The parameter information 34 of a respective treatment data set 32 comprises a plurality of objectifiable parameters indicative of a pathological condition of the bone 46. FIG. 5 schematically shows a matrix representation of the plurality of parameters for the preceding treatment interventions in a matrix representation 54.

It is favorable if, for example, at least around 20 parameters are used, preferably 40 parameters or more.

In preceding treatment interventions, the respective patient 52 is treated according to a treatment type from a group of preferably predefined treatment types. FIG. 5 shows this as an example in a matrix representation 56, which shows the treatment types linked to the preceding treatment interventions. The treatment information 36 of each treatment data set 32 is indicative of the conducted treatment type for the patient 52. As an example, FIG. 5 shows a group of five different treatment types.

In addition, the success information 38 of a respective treatment data set 32 is utilized. A success assessment for a respective treatment intervention is shown in a matrix representation 58. As an example, FIG. 5 shows six different classifications for the success information.

In order to determine the objectifiable parameters of the parameter information 34, an initial data set 62 of the bone 46 considered to be defective is first created using the CT device 30 during a preoperative data acquisition 60 in particular. In the case of revision surgery, the initial data set 62 contains contributions from the implant 42. In particular, the initial data set 62 comprises a CT data set and/or an X-ray image with a 3D representation of the bone 46. The initial data set 62 is indicative of an actual state of the bone 46 and serves as a basis for the data processing device 12 to provide at least some of the parameters.

The initial data set 62 may be provided to the data processing device 12 by the CT device 30 automatically and/or by a user.

The data processing device 12 is configured and programmed in such a way that it performs a segmentation 64 of the initial data set 62. In the case of revision surgery, contributions attributable to the implant 42 in the initial data set 62 are computationally removed by the data processing device 12, whereby an adapted initial data set 66 is created without contributions from the implant 42. In the case of non-revision interventions, but first-time implantation of an implant 42, this step of computational removal can be omitted and the segmented initial data set 62 can be used directly by the data processing device 12.

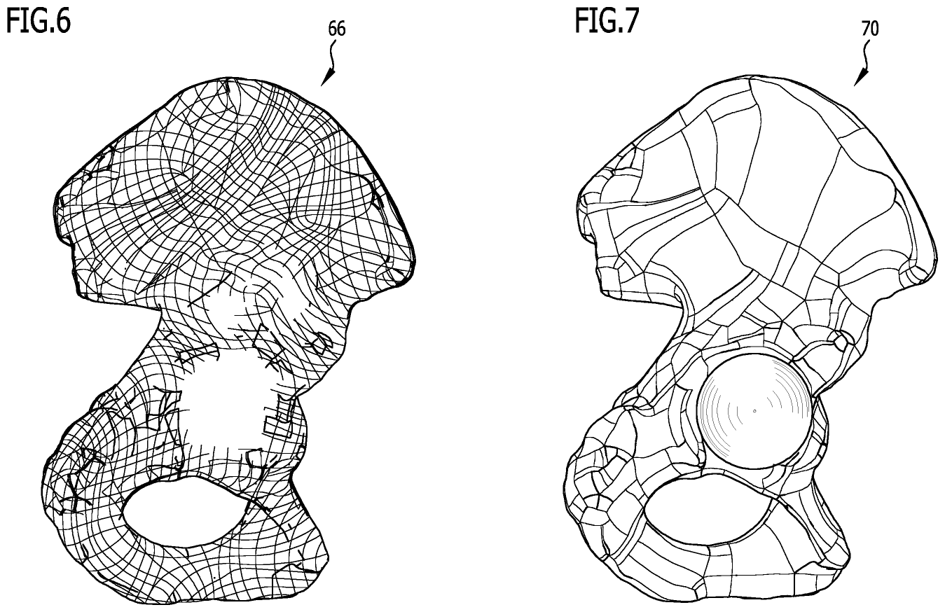

FIG. 6 schematically shows a 3D representation of the bone 46 in the adapting initial data set 66.

Subsequently, the data processing device 12 computationally determines a healthy-state data set of the bone 46 of the patient 52 based on the initial data set, in this case the initial data set 66. This step is marked in FIG. 5 with the reference sign 68. FIG. 7 shows a 3D representation of the bone 46 in the healthy-state data set 70.

In order to calculate the healthy-state data set, for example, a statistical form model may be used for the bones 46. A statistically probable healthy situation of the bone 46 (native situation) is calculated.

By comparing the healthy-state data set 70 with the initial data set 66, the parameters and thus the parameter information 34 can be provided. The parameters are at least partially objectifiable in order to be able to record bone defects quantitatively. The reference sign 72 in FIG. 5 indicates the corresponding defect analysis.

The parameter information 34 is stored in the respective treatment data set 32, as symbolized by the matrix representation 54.

The user, in particular the surgeon, can select a suitable treatment type for the patient 52, taking into account the parameter information 34. The selected treatment 74 can be selected from the selection of the group of treatment types. The reference sign 76 indicates the selection of a treatment type for a respective treatment intervention as symbolized according to the matrix representation 56.

The data processing device 12 is configured to relate the treatment types and the parameter information to each other. This is shown in FIG. 5 using the matrix representations 78. This shows exemplary tables which, grouped according to the treatment type, reflect the respective treatment interventions and the corresponding parameter information 34. In particular, a table 80 may be assigned to each treatment type.

It is understood that this visualization is only exemplary and that, depending on the data structures used in the memory device 14, a different type of storage and/or visualization of the data could be provided.

Postoperatively, the user may carry out a success assessment 82 for the preceding intervention. Data inputs from the user for the indication of the treatment 74 and the success assessment 82 may be carried out in particular via the input devices 16 and/or 24. It may be advantageous if a user application program (for example an app) that can be used by the user is executably stored on the additional devices 26, via which the data inputs can be received and then transmitted from the additional device 26 to the data processing device 12. In particular, the use of additional devices 26 offers the possibility of including the patients 52 in the success assessment 82.

Based on the data input, the data processing device 12 generates the success information via an analysis 84, as shown by way of example in the matrix representation 58.

As FIG. 5 further schematically illustrates, the data processing device 12 is configured and programmed to create a quantitative relationship 86 based on preceding treatment interventions. In particular, the quantitative relationship 86 is created between characteristic features of a respective treatment type and at least a part of the parameters of the parameter information 34. The data processing device 12 includes the respective success information 38 in the quantitative relationship 86.

In particular, the data processing device 12 thus creates a static model comprising information about the main characteristics of bone defects in each treatment type and indications about the success of the selected treatment type.

The data processing device 12 is configured and programmed in such a way that it preferably creates the quantitative relationship 86 independently. Accordingly, the data processing device 12 may be configured and programmed in a self-learning manner to create the quantitative relationship 86 without user support. For example, a machine learning algorithm and/or a neural network is used to create the relationship 86.

Reference sign 88 is an example of a diagram of a PC analysis (PCA, Principal Component Analysis). A PC analysis may be used to identify the characteristic features of the respective treatment type and is performed, for example, using the machine learning algorithm mentioned above or the neural network.

The database with the treatment data sets 32 of the system 10 may be expandable. The parameter information 34, the treatment information 36 and the success information 38 for a respective treatment intervention may be supplemented, evaluated by the data processing device 12 and may be used to improve the quantitative relationship 86.

For an upcoming treatment intervention (FIG. 8), the parameter information 34 is generated based on the defect analysis 72 for the bone 46 to be treated.

The data processing device 12 can determine at least one selected treatment type from the group of treatment types based on the provided parameter information 34 and the quantitative relationship 86.

Since the relationship 86 links success information 38 with parameter information 34 of preceding treatment interventions as well as the respective treatment type, the data processing device 12 is capable of determining the selected treatment type with regard to a (presumably) successful treatment intervention. The determination of the at least one treatment type is marked with the reference sign 90.

Based on this, the data processing device 12 can provide the user at the notice device 18 with a treatment suggestion about the at least one selected treatment type, as indicated in FIG. 8 by the reference sign 92.

The disclosure offers the advantage that the treatment suggestion 92 can be made based on previous experience. This experience of preferably well configured surgeons can be advantageously used for future treatment interventions, and users can be supported in these treatment interventions.

The user, in particular the surgeon, can accept the treatment suggestion 92. Alternatively, the user can decide on a different treatment type in deviation from the treatment suggestion.

If the user decides on a treatment type, the data processing device 12 can transmit the relevant provision information to the stockage device 22. Based on this, the stockage device 22 can provide the toolset 40 with the implant 42 suitable for the treatment type. Information in this regard may be stored in the treatment data set 32. If applicable, the stockage device 22 may automatically trigger a reorder of the assigned toolset 42.

It is favorable if the data processing device 12 determines a probability for a successful treatment intervention for the selected treatment type. The treatment suggestion 92 may preferably comprise an indication of the probability for the successful treatment intervention.

Furthermore, it may be provided that the data processing device 12 determines two or more selected treatment types from the group of treatment types. The treatment suggestion 92 may comprise the indication of the two or more selected treatment types.

Preferably, in the latter configuration examples, the treatment suggestion comprises an indication of the respective probability for a successful treatment intervention for at least two of the selected treatment types.

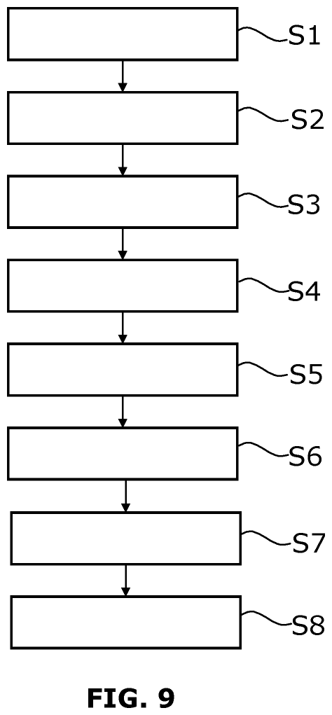

FIG. 9 shows a flow diagram of a selection method according to the disclosure. The method comprises the following steps. In step S1, a treatment data set of preceding treatment interventions is extracted from a memory device by the data processing device 12, wherein the treatment data set comprises in particular parameter information 34, treatment information 36 and/or success information 38 of the preceding treatment interventions. In step S2, relevant parameters of respective treatment types, which are decisive for a success of the respective treatment type, are determined by the data processing device 12 from the treatment data set via a static model. Thus, a correlation between successful treatment type and a selection quantity of (relevant) parameters that significantly influence the successful treatment type is determined. In particular, the statistical model may be a principal component analysis. In step S3, the data processing device 12 reads in an initial data set 62 or an actual-state data set. The initial data set 62 is in particular the CT data set of the bone 46. In step S4, a target-state data set or healthy-state data set 70 is calculated by the data processing device 12 on the basis of the actual-state data set (initial data set 62). In particular, the healthy-state data set 70 is calculated using a molded bone model.

In step S5, the data processing device 12 determines differences or deviations of parameters/parameter information 34 by comparing the healthy-state data set 70 and the initial data set 62. In step S6, the determined differences or deviations with respect to the determined relevant parameters are filtered by the data processing device 12. In step S7, the filtered differences of the parameters are compared with the corresponding ones of the historically successful treatment interventions. Furthermore, a quantity of historical treatment data is determined in which the differences are similar to each other or in particular within a (predetermined) tolerance range, for example in which a sum of the relative differences of the relevant parameters is as small as possible). In step S8, the determined quantity of historical successful treatment data is output by the notice device 18.

1.-15. (canceled)

16. A medical technology system for use in a treatment of a bone with an implant, the medical technology system comprising:
a data processing device;
a memory device; and
a notice device,
wherein in the memory device, based on a plurality of preceding treatment interventions, the following is stored linked to each other and assigned or assignable to a respective treatment intervention in a treatment data set:
parameter information comprising a plurality of objectifiable parameters that are indicative of a pathological condition of the bone;
treatment information that is indicative of a conducted treatment type for the patient for the treatment intervention, from a group of preferably predefined treatment types; and
success information that is indicative of the success of the treatment intervention,
wherein the data processing device is configured to determine relevant parameters for treatment types from the treatment data set via a static model, said relevant parameters being decisive for success of said treatment types, and
wherein the data processing device, for an upcoming treatment intervention, compares for the relevant parameters values of the upcoming treatment intervention with the corresponding values of the plurality of preceding treatment interventions,
wherein the data processing device determines at least one selected treatment type from the group of treatment types with respect to a successful treatment intervention, and
wherein the data processing device provides a treatment suggestion about the at least one selected treatment type to a user at the notice device.

17. The system according to claim 16, wherein a deviation between the values of the upcoming treatment intervention and the corresponding values of the preceding treatment interventions for the determined relevant parameters lie within a predetermined tolerance range.

18. The system according to claim 16, wherein the system comprises an analysis device for providing an initial data set that is indicative of an actual state of the bone, and
wherein the data processing device is configured and programmed to determine at least a part of the values and the objectifiable parameters for providing the parameter information on the basis of the initial data set.

19. The system according to claim 18, wherein the data processing device is configured and programmed to computationally determine a healthy-state data set of the patient based on the initial data set.

20. The system according to claim 19, wherein the data processing device determines the at least part of the values by comparing the healthy-state data set with the initial data set or the initial data set after computational removal of the implant.

21. The system according to claim 16, wherein the data processing device is configured and programmed to computationally remove an implant existing in the bone from the initial data set for the actual state of the patient.

22. The system according to claim 16, wherein the data processing device is configured and programmed to determine the at least one selected treatment type linked with a probability for a successful treatment intervention and that the treatment suggestion to the user comprises an indication of the probability.

23. The system according to claim 16, wherein the data processing device is configured and programmed to determine two or more selected treatment types from the group of treatment types and to provide the treatment suggestion about the two or more selected treatment types to the notice device.

24. The system according to claim 16, wherein the data processing device is configured and programmed to create a quantitative relationship between characteristic features of a respective treatment type and at least selected parameters of the parameter information for treatment interventions.

25. The system according to claim 24, wherein the system uses a machine learning algorithm and/or a neural network of the data processing device to create the quantitative relationship.

26. The system according to claim 24, wherein the data processing device is configured and programmed to feature the success information in this quantitative relationship for the respective treatment intervention and uses this quantitative relationship as the basis for determining the at least one selected treatment type,
wherein values of parameters that match the corresponding values of the upcoming treatment within a predetermined or predeterminable threshold are predominantly decisive parameters for the suggestion.

27. The system according to claim 25, wherein the selected treatment type is selected if the treatment intervention with the selected treatment type was successful.

28. The system according to claim 24, wherein the data processing device is configured to be self-learning and programmed to create the quantitative relationship without user intervention.

29. The system according to claim 16, wherein the system comprises at least one input device for receiving data input from a user after completion of the treatment intervention, and
wherein the data processing device is configured and programmed to create the success information based on the data input and to store it together with the parameter information and the treatment information in the treatment data set.

30. The system according to claim 29, wherein the input device is at least one portable additional device arranged spatially remote from the data processing device, or
wherein the at least one additional device comprises the input device,
wherein a user application program is stored executably on the additional device, and
wherein the data input of the user can be transmitted from the additional device to the data processing device via a communication connection.

31. The system according to claim 16, wherein the static model is a principal component analysis.

32. A computer-implemented selection method comprising the steps of:
reading of a treatment data set of preceding treatment interventions from a memory device by a data processing device;
determining a correlation between successful treatment types and a selection quantity of relevant parameters from the treatment data set by a static model, wherein the relevant parameters significantly influence the successful treatment type;
reading of an initial data set by the data processing device;
calculating a healthy-state data set from the initial data set by the data processing device;
determining differences or deviations of parameter information by comparing the healthy-state data set with the initial data set by the data processing device;
filtering of the determined differences or deviations with regard to the determined relevant parameters by the data processing device;
comparing the filtered differences with those corresponding to historically successful treatment interventions, and determining a quantity of historical treatment data for which the differences lie within a predetermined tolerance range; and
outputting the determined quantity of historical successful treatment data, in particular the treatment information, by a notice device.

33. The computer-implemented selection method according to claim 32, wherein the treatment data set comprises parameter information, treatment information and/or success information of the preceding treatment interventions and/or the data processing device calculates a healthy-state data set from the initial data set with the help of a molded bone model and/or the determined quantity of historical successful treatment data is the treatment information.

34. The computer-implemented selection method according to claim 32, wherein the treatment information comprises a group of treatment types for a treatment intervention, the parameter information comprises parameters for the treatment interventions and the success information comprises a success of the preceding treatment interventions, and the treatment data set links the respective treatment types with the parameters and the success of the treatment intervention.

35. A computer-readable storage medium comprising commands which, when executed by a computer, cause the computer to execute the method steps of the selection method according to claim 32.
